(19) United States
(12) Patent Application Publication
(10) Pub. No.: US 2026/0314935 A1
(43) Pub. Date: Oct. 15, 2026

(54) Method and Apparatus for Dental Imaging to Assess Periodontal Disease

(71) Applicant: Debra Dencek, Phoenix, AZ (US)

(72) Inventor: Debra Dencek, Phoenix, AZ (US)

(21) Appl. No.: 19/643,313

(22) Filed: Apr. 9, 2026

Related U.S. Application Data
(60) Provisional application No. 63/785,745, filed on Apr. 9, 2025.

(51) Int. Cl. A61B 5/00

(52) U.S. Cl. CPC A61B 5/0088; A61B 5/0086; A61B 5/682; A61B 2562/0233; A61B 2562/043; A61B 2576/02

(57) ABSTRACT

An improved system and method for detecting periodontal disease includes a mouthpiece for insertion into the mouth and a controller to perform imaging of the periodontia of an individual. Embedded within the walls of the mouthpiece are light sources. The light sources emit light onto the teeth and gingival tissue from both the buccal (side facing the cheek) and lingual (side facing the tongue) aspects of the mouthpiece. Receivers detect light reflected back from the tissue being imaged. The light received is compared to a reference light source. The reflected light may be utilized to generate a three-dimensional representation of the tissue surrounding the tooth. The three-dimensional representation of the gingival tissue is used to diagnose the depth of the gingival sulcus, bone level, and gingival margin around the entire tooth by which a level of periodontal disease may be determined.

### CROSS-REFERENCE TO RELATED APPLICATIONS

This application claims priority to U.S. Provisional Application Ser. No. 63/785,745, filed Apr. 9, 2025, the entire contents of which is incorporated herein by reference.

### BACKGROUND OF THE INVENTION

### Field of the Invention

The subject matter disclosed herein relates to dental imaging for use in assessing periodontal disease. More specifically, a mouthpiece is provided by which optical coherence tomography is used to provide images of periodontal structures.

### Description of Related Art

As is known to those skilled in the art, the primary technique for diagnosis of periodontal disease has changed little over the last sixty years. The common process for diagnosing periodontal disease includes a dental probe being inserted at the base of the tooth and the surrounding gingival tissue to measure the depth of the gingival sulcus and the Clinical Attachment Level (CAL). The gingival sulcus is the space between each tooth and the gum tissue which forms a small, V-shaped groove around the base of each tooth. When the gum tissue is healthy, the gingival sulcus is snug around the base of the tooth up to the point the tooth emerges from the gums, and the depth of the gingival sulcus will range between one and three millimeters (1-3 mm). If the gum tissue becomes inflamed, the gingival sulcus pulls away from the tooth, allowing food substances to more easily enter the space between the gum tissue and the tooth. The depth of the gingival sulcus can then extend to a range of more than three to four millimeters (3-4 mm) and in advanced cases up to ten millimeters (10 mm) or more. The dental probe includes markings around the tip of the probe to provide a visual indication of the depth of insertion. During a dental visit, the probe is inserted around each tooth and a depth of insertion recorded to determine a level of periodontal disease present.

These periodontal examinations are not without certain drawbacks. The procedure can be uncomfortable to a patient, particularly to a patient experiencing periodontal disease. Insertion of the probe into inflamed gum tissue can cause pain and bleeding along the gum line. Further, probing is performed at finite locations around a tooth. Small areas of detachment, which may provide an early indication of periodontal disease, are easily missed during the examination. The procedure is time consuming and increases the length of dental appointment. In addition, the expertise and knowledge of the dental professional to insert the probe at the proper angle and pressure and to properly read and understand the markings on the probe impact the accuracy of the examination.

Over time, numerous other technologies for performing the periodontal examination have been introduced in an attempt to provide both a more pleasant patient experience and a more thorough examination. Examples of these technologies include: a probe with a piezo electric sensor; an electronic probing system, referred to as a Florida probe, an ultrasonic probe; and digital probe which exerts a constant amount of pressure. Each of the probes has advantages and disadvantages. While there has been some adoption of the different styles of probes, the most prevalent method for performing periodontal examination remains the manual probe discussed above. Further, each of the different probes still requires a dental professional to manually position and insert the probe at various locations within a patient's mouth.

Thus, it would be desirable to provide an improved system and method for detecting periodontal disease.

It is further desirable to provide an improved system and method for detecting periodontal disease which improves comfort and which improves accuracy of the examination.

It would also be desirable to provide an improved system and method for detecting periodontal disease which eliminates the need for a dental professional to manually position a probe at various locations within a patient's mouth.

### BRIEF DESCRIPTION OF THE INVENTION

The subject matter disclosed herein describes an improved system and method for detecting periodontal disease. A mouthpiece is inserted into the mouth. Embedded within the walls of the mouthpiece are light sources. The light sources emit light onto the teeth and gingival tissue from both the buccal (side facing the cheek) and lingual (side facing the tongue) aspects of the mouthpiece. Receivers detect light reflected back from the tissue being imaged. The light received is compared to a reference light source. Because the emitted light reflects off different tissue in different manners, the reflected light may be utilized to diagnose both the hard and soft tissue in the mouth, such as the alveolar bone or the gingival tissue. Further, a three-dimensional representation of the gingival tissue surrounding the tooth may be generated. Using the three-dimensional representation of the gingival tissue, the depth of the gingival sulcus, bone level, and gingival margin, also referred to as the Clinical Attachment Level (CAL), around the entire tooth may be determined and measured to provide an improved system and method for detecting periodontal disease. The mouthpiece eliminates probing of the gingival tissue thereby eliminating the need for a dental professional to manually position a probe at various locations within a patient's mouth and providing improved patient comfort during the procedure and providing improved accuracy of the examination. In addition, the ability of an individual to utilize the system at home permits more frequent monitoring rather than relying on a once or twice a year examination at a dental office.

According to one embodiment of the invention, an apparatus for assessing periodontal disease includes a mouthpiece and a controller. The mouthpiece includes a buccal wall, a lingual wall, and a bite portion extending between the buccal wall and the lingual wall. The buccal wall has multiple first near-infrared (NIR) light emitting sources and multiple first light receiving elements. The lingual wall has multiple second NIR light emitting sources and multiple second light receiving elements. The controller is configured to selectively energize the first NIR light emitting sources to emit NIR light toward the lingual wall and sample a value of NIR light from the first light receiving elements/The NIR light received by the first light receiving elements is emitted from the first NIR light emitting sources and reflected from either tissue or teeth in the mouthpiece. The controller selectively energizes the second NIR light emitting sources to emit NIR light toward the buccal wall and samples a value of the NIR light from the second light receiving elements. The NIR light received by the second light receiving elements is emitted from the second NIR light emitting sources and reflected from either the tissue or the teeth in the mouthpiece. An image of the tissue and the teeth is generated as a function of the value of NIR light received from the first and the second light receiving elements.

According to aspects of the invention, the controller is further configured to identify at least one of: a top of enamel, a cementoenamel junction, a gingival margin, a crestal bone, a periodontal ligament, and a gingival sulcus corresponding to at least one tooth from the image generated. The controller is further configured to identify a level of periodontitis for the at least one tooth as a function of a depth of the gingival sulcus and to identify the level of periodontitis at a plurality of locations around the at least one tooth.

According to another aspect of the invention, the first plurality of NIR light emitting sources may be NIR light-emitting diodes spaced apart along an inner surface of the buccal wall, and the second plurality of NIR light emitting sources may be NIR light-emitting diodes spaced apart along an inner surface of the lingual wall. Optionally, the buccal wall may include multiple first NIR transceivers, where each of the first NIR transceivers includes one of the first NIR light emitting sources and one of the first light receiving elements. The lingual wall may include multiple second NIR transceivers, where each of the second NIR transceivers includes one of the second NIR light emitting sources and one of the second light receiving elements.

According to still another aspect of the invention, the apparatus includes a bundle of fiberoptic strands extending between the controller and the mouthpiece. The first NIR light emitting sources may be a first portion of fiberoptic strands having a distal end at an inner surface of the buccal wall, and the second NIR light emitting sources may be a second portion of fiberoptic strands having a distal end at an inner surface of the lingual wall. The apparatus may also include a light source in the controller, where the light source is configured to generate NIR light and provide the NIR light to a proximal end of the first portion and the second portion of fiberoptic strands. A beam splitter may be provided between the light source and the proximal end of the first and the second portion of fiberoptic strands. The beam splitter divides the NIR light between the fiberoptic strands and a reference, optical conductive path, and the beam splitter combines NIR light reflected from both the fiberoptic strands and the reference optical conductive path into the NIR light sampled from the first and the second light receiving elements.

According to yet another aspect of the invention, the NIR light from the light source is delivered to the proximal end of the first and the second fiberoptic strands. A portion of the NIR light is reflected from an air-cable barrier at the distal end of the first and the second portion of the fiberoptic strands, and the controller is further configured to sample the portion of the NIR light reflected as a reference value for the NIR light.

According to another embodiment of the invention, a method for assessing periodontal disease includes emitting a near-infrared (NIR) light from multiple first NIR light emitting sources located on a buccal wall of a mouthpiece toward a lingual wall of the mouthpiece and receiving the NIR light from the first NIR light emitting sources at multiple first light receiving elements located on the buccal wall of the mouthpiece when the NIR light is reflected from either tissue or teeth in the mouthpiece. A near-infrared (NIR) light is emitted from multiple second NIR light emitting sources located on the lingual wall of the mouthpiece toward the buccal wall of the mouthpiece. The NIR light is received from the second NIR light emitting sources at multiple second light receiving elements located on the lingual wall of the mouthpiece when the NIR light is reflected from either the tissue or the teeth in the mouthpiece. An image of the tissue and the teeth is generated as a function of the NIR light received from the first and the second light receiving elements.

These and other advantages and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

Various exemplary embodiments of the subject matter disclosed herein are illustrated in the accompanying drawings in which like reference numerals represent like parts throughout, and in which:

FIG. 1 is an exemplary environment incorporating one embodiment of the invention ;

FIG. 2 is an exemplary environment incorporating another embodiment of the invention;

FIG. 3 is an elevational view of a mouthpiece and a controller of FIG. 1;

FIG. 4 is a partial sectional view of the mouthpiece of FIG. 1;

FIG. 5 is another exemplary environment incorporating still another embodiment of the invention;

FIG. 6 is an elevational view of a mouthpiece and a controller of FIG. 5;

FIG. 7 is a block diagram representation of one embodiment of a mouthpiece and controller for use in assessing periodontal disease;

FIG. 8 is a block diagram representation of another embodiment of a mouthpiece and controller for use in assessing periodontal disease;

FIG. 9 is a block diagram representation of another embodiment of a mouthpiece and controller for use in assessing periodontal disease;

FIG. 10 is a block diagram representation of another embodiment of a mouthpiece and controller for use in assessing periodontal disease; and

FIG. 11 is a sectional view of a tooth and surrounding tissue illustrating a difference between healthy and diseased tissue.

In describing the various embodiments of the invention which are illustrated in the drawings, specific terminology will be resorted to for the sake of clarity. However, it is not intended that the invention be limited to the specific terms so selected and it is understood that each specific term includes all technical equivalents which operate in a similar manner to accomplish a similar purpose. For example, the word “connected,” “attached,” or terms similar thereto are often used. They are not limited to direct connection but include connection through other elements where such connection is recognized as being equivalent by those skilled in the art.

### DETAILED DESCRIPTION OF THE INVENTION

The various features and advantageous details of the subject matter disclosed herein are explained more fully with reference to the non-limiting embodiments described in detail in the following description.

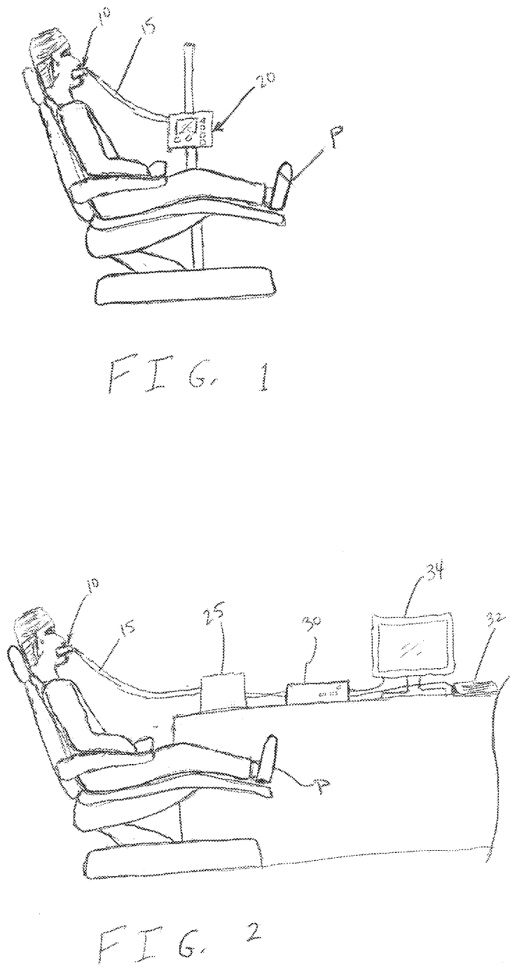

Turning initially to FIG. 1, a patient, P, is illustrated in a dental chair. A mouthpiece 10 is connected to a controller 20 via a cable 15. According to the illustrated embodiment, the controller 20 is configured to mount to a pole with a wheeled base. The wheeled base allows the controller 20 to be positioned around the dental chair in a location convenient for the patient, P, and for a dental professional, such as a dental hygienist or a dentist, to perform the procedure. Optionally, the controller 20 may be configured to rest on a cart, which may be similarly positioned in a convenient location. With reference also to FIG. 2, another embodiment of the controller 25 may be configured to rest on a counter. The controller 25 is in communication with a computer, where the computer includes a processing unit 30, a keyboard 32, and a display 34. The cable 15 is of sufficient length to extend between the controller 25 and the patient, P, sitting in the dental chair. According to yet another embodiment of the invention, an application running on a mobile device including, but not limited to, a laptop computer, a tablet, a notebook, or a smartphone may be in communication with the controller 25 to receive and display images from the controller 25.

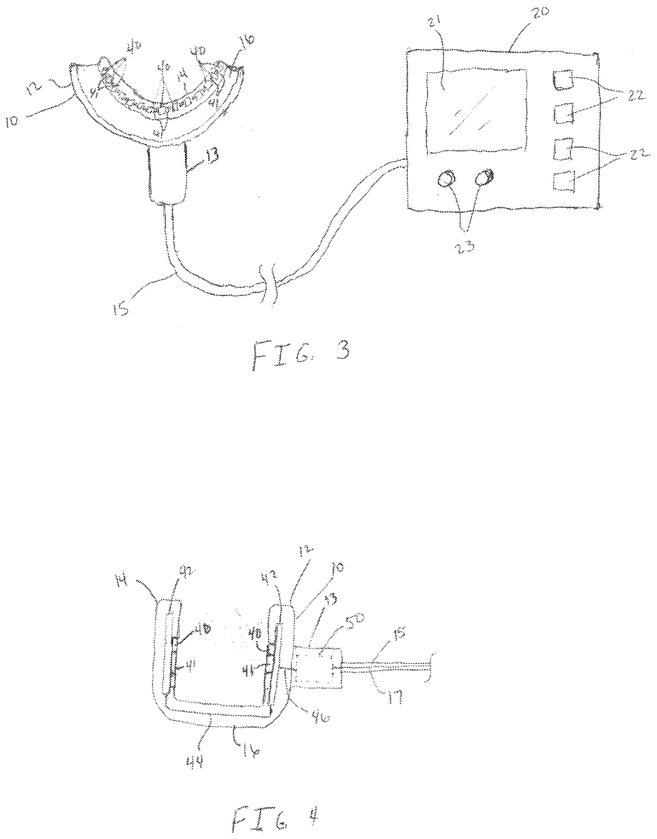

With reference also to FIG. 3, the controller 20 and mouthpiece 10 of FIG. 1 are shown in additional detail. The controller 20 may include buttons 22, dials 23, or other input devices, such as a touchscreen, switch, slider, or any combination thereof to receive input from a dental professional performing the examination. The combination of input devices will be referred to herein as a user interface 25. A display 21 is provided to provide information to the dental professional. The combination of display 21 and user interface 25 allow the dental professional to adjust settings in the controller 20 to perform the examination.

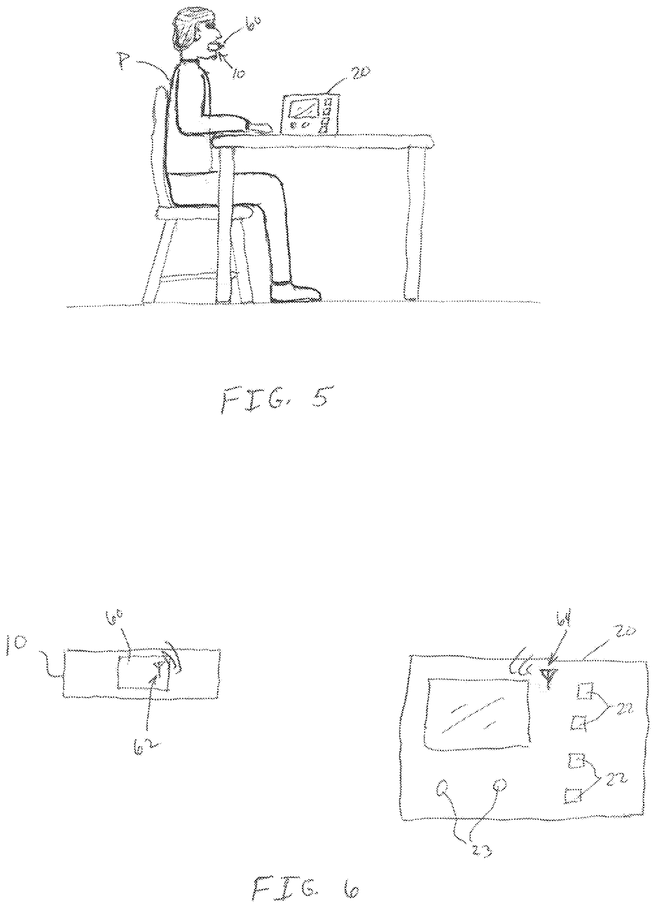

With reference next to FIG. 5, it is also contemplated that the controller 20 may be used in a home environment. A patient, P, is illustrated sitting at a table in a home. The mouthpiece 10 includes a wireless module 60 mounted on the mouthpiece for communication with the controller 20. As further shown in FIG. 6, both the wireless module 60 and the controller 20 include antennas 62, 64 for communication between the devices. It is contemplated that the wireless module 60 and controller 20 may be configured to communicate with each other on a personal area network (PAN) utilizing, for example, a protocol such as Adaptive Network Topology (ANT), Ultra-wideband (UWB), Bluetooth, Bluetooth Low Energy (BLE), Z-Wave, Zigbee, or Wireless Universal Serial Bus (USB). Optionally, a wireless local area network (WLAN) maybe established for Wi-Fi communication, for example, via the IEEE 802.11 family of protocols. It is further contemplated that wired or wireless versions of the mouthpiece 10 and controller 20 may be utilized either in a medical or dental environment or in a remote environment, such as at a home of the patient. Instructions may be provided to a patient, P, such that the patient, or another person assisting the patient, is able to set up and operate the controller 20 to provide an at-home periodontal examination.

Turning again to FIG. 3, the mouthpiece 10 is generally u-shaped and curved to fit either the maxillary (upper) teeth or mandibular (lower) teeth of a patient. The mouthpiece includes a buccal wall 12 and a lingual wall 14 with a bite portion 16 extending between the buccal wall 12 and the lingual wall 14. The entire mouthpiece 10 is curved to fit within a patient's mouth and to follow either the maxillary or mandibular teeth. A housing is formed over the buccal wall 12, lingual wall 14, and bite portion 16 to define a continuous mouthpiece 10. According to one aspect of the invention, the mouthpiece 10 may be custom made to conform to an individual's mouth formed in different fixed sizes to accommodate patient's having different size mouths. According to another aspect of the invention, the mouthpiece may be formed of a resilient material permitting some flexibility in the mouthpiece to conform to the size and shape of different mouths.

The mouthpiece 10 includes multiple light emitting sources 40 and light receiving elements 41 along both the buccal wall 12 and the lingual wall 14 of the mouthpiece. Including the light emitting sources 40 and light receiving elements 41 along both walls permits scanning of both sides of the teeth in tandem. For ease of illustration in FIG. 3, the light emitting sources 40 and light receiving elements 41 are illustrated just along the lingual wall 14. According to the embodiment illustrated in FIG. 3, the light emitting sources 40 are multiple light emitting diodes (LEDs) embedded within the mouthpiece. Preferably, the LEDs 40 are near infrared (NIR) LEDs. NIR LEDs typically emit electromagnetic radiation having a wavelength between about seven hundred and two thousand five hundred nanometers (700-2500 nm). Electromagnetic radiation in this wavelength is at the border of the visual light spectrum. NIR LEDs at the seven hundred nanometer wavelength fall in the range of red visible light. As the wavelength increases to about eight hundred or eight hundred fifty nanometers (800-850 nm), the electromagnetic radiation becomes invisible, or may be a faint glow, to the human eye. Electromagnetic radiation above nine hundred fifty nanometers (950nm) is virtually invisible to the human eye. According to one aspect of the invention, NIR light at about one thousand three hundred nanometers (1300 nm) is selected.

Electromagnetic radiation in the NIR spectrum has a further advantage of penetrating into materials and/or tissue further than light in the visible spectrum without causing damage to, or significant heating of, the tissue. Thus, electromagnetic radiation in the NIR spectrum is useful for imaging the hard, soft, and connective tissues of the patient's mouth. Each NIR LED 40 emits radiation in the desired spectrum. The radiation travels toward the patient's tooth and other oral tissue, such as bone, blood, ligaments, or gum tissue. The radiation penetrates for at least one millimeter and preferably two millimeters into tissue and is reflected back toward the mouthpiece. The mouthpiece further includes receivers 41 interspersed with the LEDs 40 to receive the radiation reflected back from the patient's tooth and other oral tissue. Optionally, the LEDs 40 and receivers 41 may be integrally formed as a transceiver able to both transmit and receive.

With reference also to FIG. 4, the NIR LEDs 40 and receivers 41 are mounted to a circuit board 42. A first circuit board 42 is present on the buccal side 12 of the mouthpiece 10, and a second circuit board 42 is present on the lingual side 14 of the mouthpiece. Each circuit board 42 may be made of a flexible material to follow the curvature of the mouthpiece 10. Connections 44 extend between the first and second circuit boards 42 via the bite portion 16 of the mouthpiece 10. The connections 44 may include electrical connections, optical connections, mechanical connections, or a combination thereof. The illustrated mouthpiece 10 is formed to image only the upper teeth or the lower teeth at one time. The mouthpiece 10 may be inserted into the patient's mouth in a first orientation to obtain an image of the upper teeth and then inserted into the patient's mouth in the opposite orientation to obtain an image of the lower teeth. Optionally, a second set of circuit boards 42 may be provided or the first set of circuit boards, as shown, may extend both upwards and downwards from the bite portion 16 of the mouthpiece 10 such that both the upper and lower teeth may be imaged with a single insertion of the mouthpiece into the patient's mouth.

A junction box 13 may be mounted to or integrally formed with the mouthpiece 10. The junction box 13 provides an interface between the cable 15 and the mouthpiece 10. An optional control circuit 50 is illustrated within the junction box 13, where the control circuit 50 may provide routing, switching, and/or processing of signals provided to or received from the mouthpiece 10 or controller 20. Additional connectors 17 extend within the cable 15 between the junction box 13 and the controller 20. These additional connectors 17 may be electrical connectors, optical connectors, or a combination thereof. In the wireless example of FIG. 5, the wireless module 60 may replace or be incorporated into the junction box 13, providing a wireless connection between the mouthpiece 10 and the controller 20.

According to one aspect of the invention, the first and second circuit boards 42, the connections 44 therebetween, and the connections 46 to junction box 13 are made, and a plastic or rubber housing is molded over the components to form the mouthpiece 10. The junction box 13 and cable 15 may similarly be formed as a single component. Complementary connectors may be provided on the mouthpiece 10 and junction box 13 to allow the mouthpiece to be plugged into the junction box 13. Optionally, the junction box 13 may also be integrally formed with the mouthpiece 10 and a cable 15 provided which plugs into the junction box. According to yet another option, the entire apparatus from the mouthpiece 10, through the junction box 13, and on to the cable 15 may be formed as a continuous assembly for connection to the controller 20.

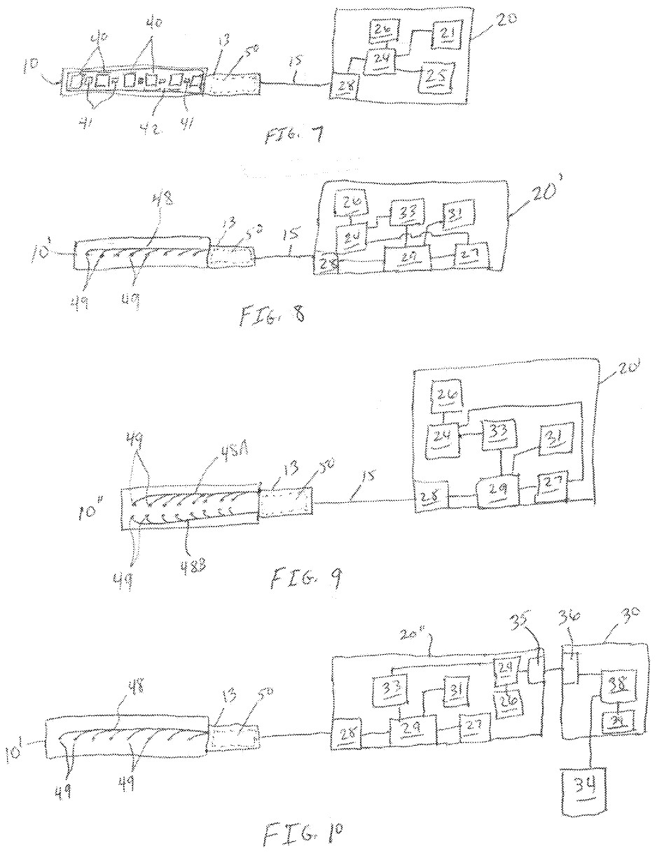

With reference next to FIG. 7, elements of the mouthpiece 10 and controller 20 are illustrated in block diagram form to illustrate interconnections. Each LED 40 receives power from and is controlled by the controller 20. The LEDs 40 are mounted to one of the circuit boards 42 within the mouthpiece 10. The circuit board 42 is connected via the junction box 13 and cable 15 to the controller 20. The controller 20 includes a communication interface 28 to receive the cable 15. The communication interface 28 includes a plug with a mating connection extending external to a housing of the controller 20. The end of the cable 15 distal from the mouthpiece 10 includes a complementary mating connection to be joined to the plug on the communication interface 28. The communication interface 28 may further include logic circuits such as buffers, amplifiers, switches, and the like to manage electrical and/or optical signals being transmitted between the controller 20 and the mouthpiece. The controller 20 further includes a processor 24 and a memory 26 in communication with the processor 24. The processor 24 may be a single device, or multiple devices executing in parallel, asynchronously, or synchronously. The memory 26 may include a single device or multiple devices, where the memory is at least in part, non-transitory to store data and instructions for desired operation of the controller 20. The processor 24 reads the instructions from the memory 26 and executes the instructions to achieve desired operation of the controller 20. The processor 24 receives input from the user interface 25 and displays data on the display 21 to facilitate operation of the controller 20.

With reference to FIG. 8, another embodiment of the mouthpiece 10′ and controller 20′ is illustrated. Rather than NIR LEDs 40, a bundle of fiberoptic strands 48 is embedded within the mouthpiece 10′. Ends 49 of individual strands of the fiberoptic bundle 48 are positioned along an interior surface of the mouthpiece 10′ such that a first portion of the ends 49 emit light toward the buccal side of a patient's teeth and other oral tissue and a second portion of the ends 49 emit light toward the lingual side of a patient's teeth and other oral tissue. The controller 20′ selectively directs light along one of the strands to emit light from the end 49 of the strand. The controller 20′ may then disable transmission of light and utilize the bundle of fiberoptic strands 48 as a receiver as well. Light reflected from the patient's teeth and other oral tissue may be received at an end 49 of a strand and conducted back to the controller 20′. With the bundle of fiberoptic strands 48 a light source other than the LEDs 40 embedded in the mouthpiece 10, as described in the initial embodiment, is required. The controller 20′ illustrated in FIG. 8 includes a light source 27. The light source may be, but is not limited to, a super-luminescent diode, a swept source light source, a tunable laser, or a low coherence light source. A beam splitter 29 is provided between the light source 27 and the interface 28. The interface 28 includes a connector to receive the cable 15 and is configured to transmit light from the beam splitter 29 onto fiberoptic strands in the cable 15. The beam splitter 29 also directs light from the light source 27 to a reference path 31. According to one aspect of the invention, the reference path 31 is an optical conductive path of a known length, such that light reflected from the reference path back to the beam splitter 29 travels a known distance. A mirror in the reference path 31 may be adjustable to vary a length of travel of the light along the reference path 31. After light travels from the controller 20 to the fiberoptic bundle 48 and is reflected back to the controller 20, the beam splitter 29 combines the light received from both the mouthpiece 10 and the reference path and directs the reflected light to a sensor 33. The sensor 33 generates a feedback signal for the processor 24 by which an image of the teeth and other oral tissue may be generated.

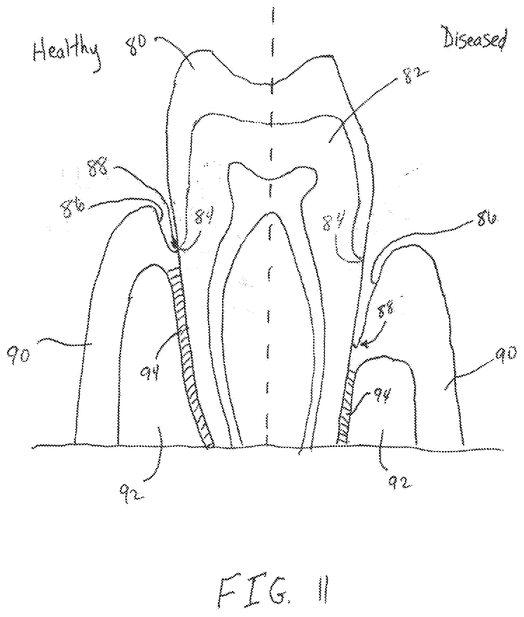

With reference also to FIG. 9, still another embodiment of the mouthpiece 10″ is illustrated. In this embodiment, the mouthpiece 10″ includes a first bundle of fiberoptic strands 48A and a second bundle of fiberoptic strands 48B. Ends 49 of individual strands from both fiberoptics bundle 48A, 48B are positioned along an interior surface of the mouthpiece 10′ such that a first portion of the ends 49 face the buccal side of a patient's teeth and a second portion of the ends 49 face the lingual side of a patient's teeth. Rather than alternately transmitting and receiving light along a single fiberoptic strand, the first fiberoptic bundle 48A is configured to emit light, and the second fiberoptic bundle 48B is configured to receive light. The controller 20′ selectively directs light along the strands of the first fiberoptic bundle 48A to emit light from the end 49 of the strand, and directs light received at an end 49 of a strand for the second fiberoptic bundle 48B to the beam splitter 29 and, in turn, to the sensor 33 within the controller 20′.

Turning next to FIG. 10, yet another embodiment of the mouthpiece 10′ and controller 20″ is illustrated. The mouthpiece 10′ includes a fiberoptic bundle 48 and ends 49 of individual strands of the fiberoptic bundle 48 are positioned along an interior surface of the mouthpiece 10′. A first portion of the ends 49 emit light toward the buccal side of a patient's teeth and other oral tissue and a second portion of the ends 49 emit light toward the lingual side of a patient's teeth and other oral tissue. The controller 20″ selectively directs light along one of the strands to emit light from the end 49 of the strand. The controller 20″ may then disable transmission of light and utilize the fiberoptic bundle 48 as a receiver as well. Light reflected from the patient's teeth and other oral tissue may be received at an end 49 of a strand and conducted back to the controller 20″. Alternately, the mouthpiece 10″ of FIG. 9 may be utilized such that separate fiberoptic bundles 48 are provided to transmit and receive light between the mouthpiece 10″ and the controller 20″. The controller 20″ includes a light source 27. The light source may be, but is not limited to, a super-luminescent diode, a swept source light source, a tunable laser, or a low coherence light source. A beam splitter 29 is provided between the light source and the interface 28. The interface 28 includes a connector to receive the cable 15 and is configured to transmit light from the beam splitter 29 onto fiberoptic strands in the cable 15. The beam splitter 29 also directs light from the light source 27 to a reference path 31. According to one aspect of the invention, the reference path 31 is an optical conductive path of a known length, such that light reflected from the reference path back to the beam splitter 29 travels a known distance. A mirror in the reference path 31 may be adjustable to vary a length of travel of the light along the reference path 31. After light travels from the controller 20 to the fiberoptic bundle 48 and is reflected back to the controller 20, the beam splitter 29 combines the light received from both the mouthpiece 10 and the reference path and directs the reflected light to a sensor 33. The sensor 33 generates a feedback signal for the processor 24 by which an image of the teeth and other oral tissue may be generated.

The controller 20″ of FIG. 10 is additionally in communication with the processing unit 30 of the computer. A first communication interface 35 is present in the controller 20″ and a second communication interface 36 is present in the processing unit 30. According to the illustrated embodiment, a cable extends between the controller 20″ and the processing unit 30. Optionally, both the controller 20″ and the processing unit 30 may be configured to communicate wirelessly to each other, for example, via a wireless Wi-Fi network. The processing unit includes a processor 38 and memory 39. The processor 38 may be a single device, or multiple devices executing in parallel, asynchronously, or synchronously. The memory 39 may include a single device or multiple devices, where the memory is at least in part, non-transitory to store date and instructions for desired operation of the computer. The processor 38 reads the instructions from the memory 39 and executes the instructions to achieve desired operation of the computer. Functions described below with respect to control of the light source 27, processing of feedback signals from the sensor 33, and other functions performed to achieve images of the patient's teeth may be performed in whole or in part on the processor 24 of the controller, the processor 38 of the computing device 30, or a combination thereof.

In operation, the controller 20 and mouthpiece 10 provide an improved system and method for detecting periodontal disease. For ease of discussion, the controller will be discussed herein generally and referred to by reference numeral 20. It is understood that the discussion of the controller may refer to each embodiment of the controller, identified as 20, 20′, or 20″. Further, as illustrated in FIG. 10, a portion of the control steps of the controller 20 may be implemented in the computing device 30 in communication with the controller 20. Similarly, the mouthpiece 10 will be discussed herein generally and referred to by reference numeral 10. It is understood that the discussion of the mouthpiece may refer to each embodiment of the mouthpiece, identified as 10, 10′, or 10″.

According to one aspect of the invention, the controller 20 is configured to perform optical coherence tomography (OCT) to obtain image information of the patient's teeth and other oral tissue. OCT is a non-invasive imaging technique commonly used in ophthalmology and which is gaining use in other fields of medicine. OCT uses reflection of low-power, non-ionizing near infrared (NIR) light to provide two-dimensional (2D) and three-dimensional (3D) images of subsurface tissue. The radiation emitted, either from the NIR LEDs 40 or from the ends 49 of the fiberoptic strands, in the near infrared wavelength penetrate the tissue being imaged for varying depths and at varying rates depending on the type of tissue being imaged. With reference also to FIG. 11, tissue that may be present within the path of the emitted radiation includes, but is not limited to, the periodontal ligament 94, the gingival margin, blood flow, tartar, bone 92, and the like.

The controller 20 selectively enables a light source, i.e., the NIR LEDs 40 or the source 27 within the controller, to emit light from the mouthpiece 10 toward the teeth and gum tissue being imaged. The emitted light penetrates the tissue for a depth and is reflected back toward the interior surfaces of the mouthpiece 10. According to one aspect of the invention, the NIR light penetrates for at least one or two millimeters. The reflected light is received by the receivers 41 or by the fiberoptic strands. If the light is captured by receivers 41 in the mouthpiece, each receiver 41 generates a feedback signal corresponding to the amount of light incident on the receiver, and the feedback signal is transmitted back to the controller 20. If the light is captured by fiberoptic strands, the light is conducted back to the controller 20 and to the sensor 33 within the controller 20. The sensor 33 converts the light received into a feedback signal corresponding to the light incident on the sensor 33. The feedback signal is analyzed by the processor 24 in the controller 20 to determine the penetration of the light into the tissue being imaged.

The reflected light is analyzed to determine a delay in the time of flight between when the light is emitted and when the light is captured at the sensor. In order to determine the delay in the time of flight, it is typically necessary to compare the light reflected of the sample being imaged against a well-known reference light sample. The reference light sample is obtained by emitting the light from the same source along a reference path 31 having a known length. The light which travels along the reference path 31 is reflected by a mirror at the end of the reference path 31 back to the sensor 33. The mirror may be adjustably positioned to vary the length of the reference path 31 as desired, yet still provide a fixed length of travel for the light along the reference path 31. As illustrated in FIGS. 8-10, light from the light source 27 first is passed to a beam splitter 29. According to one aspect of the invention, the beam splitter 29 is an interferometer. The beam splitter 29, or interferometer, receives the light from the source 27 and splits the light from the source along two different paths. A first portion of the light then travels to the mouthpiece 10 and is emitted toward the patient's teeth and gum tissue. This light is reflected back and returns to the beam splitter 29. A second portion of the light is routed along the reference path 31. The second portion of the light is reflected back to the beam splitter 29 as well. The beam splitter 29 combines the reflected first and second portions of the light and transmits the combined light to the sensor 33.

When combining the light from the two different reflection paths, the light will either combine constructively or destructively. Light waveforms which are in phase with each other will combine constructively, or in an additive manner. An amplitude of light output from the beam splitter 29 will be greater in magnitude than the amplitude of either waveform entering the beam splitter 29. Light waveforms which are out of phase with each other will combine destructively, or in a subtractive manner. An amplitude of light output from the beam splitter 29 will be lower in magnitude than the amplitude of either waveform entering the beam splitter. The addition or subtraction of the two waveforms may also identify different frequencies of light present in one of the two reflected light beams. Light emitted from the light source and which travels along the reference path 31 is of a known frequency and amplitude. The reference path 31 will provide a known length of travel and little or no interference with the reference light. Thus, the reflected light, received from the reference path 31, should similarly be of a known frequency and amplitude. The portion of the light which is directed toward the mouthpiece 10 and emitted toward the patient's teeth and other oral tissue may change in frequency and amplitude as a function of the tissue with which it engages and from which it is reflected. Thus, the portion of the light received at the beam splitter 29 that has been reflected from the tissue under examination may include content of unknown frequency and amplitude. The two reflected light beams are combined and sent to the sensor 33 for digitization. The reflected NIR light is used to generate a sectional image of the tooth and oral tissue from which the NIR light was reflected. Each sectional image is obtained by performing spectral analysis of the feedback signal from the sensor 33. A Fourier analysis of the feedback signal determines the frequency content present in the feedback signal. Based on the frequency content of the feedback signal, the controller 20 determines the type of tissue from which the light was reflected.

The discussion above refers to a light source 27 present within the controller 20. With reference to the embodiment of FIG. 7, it is contemplated that one NIR LED 40 may be utilized as a reference light source. A mirrored surface may be provided over one LED 40 such that light emitted from the LED 40 is reflected back to a receiver 41. The known distance between the LED 40 and the receiver 41 may be utilized as a reference path 31. Optionally, an LED 40 may be embedded within the housing of the mouthpiece 10 with a single fiberoptic strand. The reference LED 40 may be configured to emit light onto the fiberoptic strand. The light may travel along a length of the fiberoptic strand to a receiver 41 or, optionally, the distal end of the fiberoptic strand may include a reflective coating such the light is first transmitted along the strand and then reflected back to a receiver 41. The reference LED 40 may be alternately energized and de-energized to transmit light along the fiberoptic strand and then to allow light to return to the receiver 41.

According to still another aspect of the invention, the fiberoptic strands may serve between the controller 20 and the mouthpiece 10 may serve as a reference path. A portion of light travelling along the fiberoptic strand is reflected at the distal end 49 of each strand 48 by the air-cable barrier. While a majority of the NIR light is emitted from each end 49, the portion of the light reflected by the air-cable barrier may be sampled and used as a reference signal.

The controller 20 selectively energizes LEDs 40 or selectively directs light from the source 31 along specific strands of the fiberoptic bundle 48 in order to create sectional images of the patient's gum tissue. With reference again to FIG. 11, multiple features of the patient's tooth and or surrounding tissue may be identified in each image. The enamel 80 along a top surface of each tooth may be identified. Interior portions of the tooth, such as the dentine 82 or cementum, which is an exterior layer of the dentine, may also be identified. The gingiva, or gum tissue, 90, bone 92, periodontal ligament 94, or gingival sulcus 86 corresponding to each tooth may similarly be imaged.

FIG. 11 further illustrates differences between a healthy tooth (left side of image) and a diseased tooth (right side of image). Enamel 80 is present around the upper surface of each tooth. Typically, the enamel is the exposed surface of a tooth. In healthy teeth, the enamel 80 covers the exposed surface and gum tissue 90 covers the base of the tooth. If a patient is experiencing periodontal disease, or periodontitis, the gum tissue 90 becomes inflamed and may recede along the base of the tooth, exposing at least a portion of the cementum. This, in turn, permits plaque build up in these exposed regions which can cause cavities and tooth decay. Early identification of periodontitis permits corrective action before significant damage to a tooth occurs.

A first feature that may be identified from the images generated from the reflected NIR light is the cementoenamel junction (CEJ) 84. As illustrated in FIG. 11, the CEJ 84 is typically located near the base 88 of the gingival sulcus 86, where the gingival sulcus 86 is a V-shaped pocket present between the gingiva 90, gun tissue, and the tooth. In a healthy tooth, the depth of the gingival sulcus 86 is typically between one and three millimeters. When periodontal disease is present, the bone 92 and gum tissue 90 recede. The pocket 86 between the tooth and gun tissue grows such that the base 88 of the gingival sulcus 86 may be at four to five millimeters or even greater distances below the top of the gum tissue.

Emitting the NIR light against the surface of the tooth and measuring the light reflected from the surface permits the controller 32 to reconstruct an image of the tooth and tissue corresponding to each tooth. Further, the image captures measurements along the entire surface from which the NIR light is reflected rather than at a single point as may be accomplished by a dental probe. The controller 20 determines the depth of the gingival sulcus 86 at multiple locations along each tooth to determine a level of periodontitis at each location. Thus, with a single image, the mouthpiece 10 permits rapid measuring of periodontitis along multiple locations of each tooth. The imaging eliminates the need for probing each tooth to improve patient comfort. Further, the imaging improves accuracy of an examination by providing measurements at multiple locations along each tooth. By determining the depth of the gingival sulcus 86 at multiple locations, the beginning of periodontal disease occurring at one location may be detected where it may previously have been missed if a healthy location was probed rather than the probing the location at which periodontitis was beginning to occur.

In some images, a pocket between the tooth and the gum tissue 90 may not be readily visible. The gum tissue 90 may be inflamed and swollen, pressing against the tooth. However, other markers within the tooth and surrounding tissue may be utilized to determine the depth of the gingival sulcus 86. The CEJ 84 typically corresponds to the top of the gingival sulcus. Thus, measurement of the enamel 80 and underlying dentine 82 or cementum surrounding the dentine provides a location of the CEJ 84 for each tooth. As periodontal disease progresses, the bone 92 and gum tissue 90 recede. The periodontal ligament 94 which connects the tooth to the bone 92 similarly recedes. The top of the bone 92, referred to as the crestal bone, or the top of the periodontal ligament 94 will typically correspond to the base 88 of the pocket around each tooth. As a result, identifying the CEJ 84 and either the crestal bone or top of the periodontal ligament 94 and determining the distance between the two points provides a depth of the pocket surrounding each tooth.

In addition to detecting periodontitis, the mouthpiece 10 and controller 20 of the present invention may be utilized to detect other dental conditions. Gingivitis may be detected by identifying the bone 90 and gingiva 90 surrounding each tooth. Gingivitis is an inflammation of the gum tissue 90 without the corresponding recession of bone 90. Thus, the gum tissue may extend further up the side of each tooth with gingivitis than for a healthy tooth. During a standard examination with a dental probe, the gum tissue extending up the tooth may result in a deeper pocket 86 surrounding each tooth. Without knowledge of the crestal bone, a dental professional could mistake the deeper pocket for periodontitis rather than gingivitis. However, with the image providing a height of the crestal bone, it may be observed that the deeper pocket is a result of inflammation and a proper diagnosis of gingivitis may be obtained.

It should be understood that the invention is not limited in its application to the details of construction and arrangements of the components set forth herein. The invention is capable of other embodiments and of being practiced or carried out in various ways. Variations and modifications of the foregoing are within the scope of the present invention. It also being understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

We claim:

1. An apparatus for assessing periodontal disease, the apparatus comprising:
a mouthpiece, comprising:
a buccal wall, including:
a first plurality of near-infrared (NIR) light emitting sources, and
a first plurality of light receiving elements;
a lingual wall, including:
a second plurality of NIR light emitting sources, and
a second plurality of light receiving element; and
a bite portion extending between the buccal wall and the lingual wall;
a controller configured to:
selectively energize the first plurality of NIR light emitting sources to emit NIR light toward the lingual wall;
sample a value of NIR light from the first plurality of light receiving elements, wherein the NIR light received by the first plurality of light receiving elements is emitted from the first plurality of NIR light emitting sources and reflected from either tissue or teeth in the mouthpiece;
selectively energize the second plurality of NIR light emitting sources to emit NIR light toward the buccal wall;
sample a value of the NIR light from the second plurality of light receiving elements, wherein the NIR light received by the second plurality of light receiving elements is emitted from the second plurality of NIR light emitting sources and reflected from either the tissue or the teeth in the mouthpiece; and
generate an image of the tissue and the teeth as a function of the value of NIR light received from the first and the second plurality of light receiving elements.

2. The apparatus of claim 1, wherein the controller is further configured to identify at least one of: a top of enamel, a cementoenamel junction, a gingival margin, a crestal bone, a periodontal ligament, and a gingival sulcus corresponding to at least one tooth from the image generated.

3. The apparatus of claim 2, wherein the controller is further configured to identify a level of periodontitis for the at least one tooth as a function of a depth of the gingival sulcus.

4. The apparats of claim 3, wherein the controller is further configured to identify the level of periodontitis at a plurality of locations around the at least one tooth.

5. The apparatus of claim 1, wherein:
the first plurality of NIR light emitting sources are NIR light-emitting diodes spaced apart along an inner surface of the buccal wall; and
the second plurality of NIR light emitting sources are NIR light-emitting diodes spaced apart along an inner surface of the lingual wall.

6. The apparatus of claim 1 wherein:
the buccal wall further includes a first plurality of NIR transceivers;
each of the first plurality of NIR transceivers includes one of the first plurality of NIR light emitting sources and one of the first plurality of light receiving elements;
the lingual wall further includes a second plurality of NIR transceivers; and
each of the second plurality of NIR transceivers includes one of the second plurality of NIR light emitting sources and one of the second plurality of light receiving elements.

7. The apparatus of claim 1 further comprising a bundle of fiberoptic strands extending between the controller and the mouthpiece, wherein:
the first plurality of NIR light emitting sources are a first portion of fiberoptic strands having a distal end at an inner surface of the buccal wall; and
the second plurality of NIR light emitting sources are a second portion of fiberoptic strands having a distal end at an inner surface of the lingual wall.

8. The apparatus of claim 7, further comprising a light source in the controller, wherein the light source is configured to generate NIR light and provide the NIR light to a proximal end of the first portion and the second portion of fiberoptic strands.

9. The apparatus of claim 8, further comprising a beam splitter provided between the light source and the proximal end of the first and the second portion of fiberoptic strands, wherein:
the beam splitter divides the NIR light between the fiberoptic strands and a reference, optical conductive path; and
the beam splitter combines NIR light reflected from both the fiberoptic strands and the reference optical conductive path into the NIR light sampled from the first and the second plurality of light receiving elements.

10. The apparatus of claim 8, wherein:
the NIR light from the light source is delivered to the proximal end of the first and the second portion of the fiberoptic strands;
a portion of the NIR light is reflected from an air-cable barrier at the distal end of the first and the second portion of the fiberoptic strands; and
the controller is further configured to sample the portion of the NIR light reflected as a reference value for the NIR light.

11. A method for assessing periodontal disease, the method comprising the steps of:
emitting a near-infrared (NIR) light from a first plurality of NIR light emitting sources located on a buccal wall of a mouthpiece toward a lingual wall of the mouthpiece;
receiving the NIR light from the first plurality of NIR light emitting sources at a first plurality of light receiving elements located on the buccal wall of the mouthpiece when the NIR light is reflected from either tissue or teeth in the mouthpiece;
emitting a near-infrared (NIR) light from a second plurality of NIR light emitting sources located on the lingual wall of the mouthpiece toward the buccal wall of the mouthpiece;
receiving the NIR light from the second plurality of NIR light emitting sources at a second plurality of light receiving elements located on the lingual wall of the mouthpiece when the NIR light is reflected from either the tissue or the teeth in the mouthpiece; and
generating an image of the tissue and the teeth as a function of the NIR light received from the first and the second plurality of light receiving elements.

12. The method of claim 11, further comprising the step of identifying at least one of: a top of enamel, a cementoenamel junction, a gingival margin, a crestal bone, a periodontal ligament, and a gingival sulcus corresponding to at least one tooth from the image generated.

13. The method of claim 12, further comprising the step of identifying a level of periodontitis for the at least one tooth as a function of a depth of the gingival sulcus.

14. The method of claim 13, further comprising the step of identifying the level of periodontitis at a plurality of locations around the at least one tooth.

15. The method of claim 11, wherein:
the first plurality of NIR light emitting sources are NIR light-emitting diodes spaced apart along an inner surface of the buccal wall; and
the second plurality of NIR light emitting sources are NIR light-emitting diodes spaced apart along an inner surface of the lingual wall.

16. The method of claim 11, wherein:
the buccal wall further includes a first plurality of NIR transceivers;
each of the first plurality of NIR transceivers includes one of the first plurality of NIR light emitting sources and one of the first plurality of light receiving elements;
the lingual wall further includes a second plurality of NIR transceivers; and
each of the second plurality of NIR transceivers includes one of the second plurality of NIR light emitting sources and one of the second plurality of light receiving elements.

17. The method of claim 11, wherein:
a bundle of fiberoptic strands extends between the controller and the mouthpiece;
the first plurality of NIR light emitting sources are a first portion of fiberoptic strands having a distal end at an inner surface of the buccal wall; and
the second plurality of NIR light emitting sources are a second portion of fiberoptic strands having a distal end at an inner surface of the lingual wall.

18. The method of claim 17, further comprising the steps of:
generating NIR light with a light source in the controller; and
transmitting the NIR light from the light source to a proximal end of the first portion and the second portion of fiberoptic strands.

19. The method of claim 18, further comprising the steps of:
dividing the NIR light between the fiberoptic strands and a reference, optical conductive path with a beam splitter provided between the light source and the proximal end of the first and the second portion of fiberoptic strands; and
combining the NIR light reflected from both the fiberoptic strands and the reference optical conductive path into the NIR light used to generate the image of the tissue and the teeth.

20. The method of claim 18 further comprising the step of receiving a portion of the NIR light reflected from an air-cable barrier at the distal end of the first and the second portion of the fiberoptic strands.
